(19) United States
(12) Patent Application Publication
(10) Pub. No.: US 2013/0059753 A1
(43) Pub. Date: Mar. 7, 2013

(54) Global Analysis of Serum microRNAs as Potential Biomarkers for Lung Adenocarcinoma

(71) Applicants: Lorraine O'Driscoll, Dublin (IE); Ken O'Byrne, Dublin (IE)

(72) Inventors: Lorraine O'Driscoll, Dublin (IE); Ken O'Byrne, Dublin (IE)

(73) Assignees: Holy and Undivided Trinity of Queen Elizabeth, near Dublin; The Provost, Fellows, Foundation Scholars, and the other Members of Board, of the College of the

(21) Appl. No.: 13/601,703

(22) Filed: Aug. 31, 2012

Related U.S. Application Data
(63) Continuation-in-part of application No. 13/224,212, filed on Sep. 1, 2011.

(51) Int. Cl. C07H 21/04; C12Q 1/68; C40B 30/04

(52) U.S. Cl. 506/9; 536/24.31; 435/6.11

(57) ABSTRACT

A diagnostic kit to detect lung adenocarcinoma, or to stratify patients according to expected prognosis comprising at least one oligonucleotide probe capable of binding to at least a portion of a circulating miRNA selected from the group comprising miR-556, -550, -939, -616*, -146b-3p, -30c-1*, -339-5p and -656.

### CROSS-REFERENCE TO RELATED PATENT APPLICATION

This application is a Continuation-In-Part under 35 U.S.C. §120 of U.S. patent application Ser. No. 13/224,212, filed Sep. 1, 2011, the content of which is incorporated herein by reference in its entirety.

### FIELD OF THE INVENTION

The present invention relates to the identification of biomarkers suitable for use in the diagnosis and prognosis of lung adenocarcinoma, and to diagnostic kits for use in such diagnosis.

### BACKGROUND OF THE INVENTION

Early diagnosis and the ability to predict the most relevant treatment option for individuals is essential to increase survival time for non-small cell lung cancer (NSCLC) patients. Adenocarcinoma (ADC), a subtype of NSCLC, is the single biggest cancer killer and so there is an urgent need to identify minimally-invasive biomarkers to enable its early diagnosis.

Lung cancer is the leading cause of cancer deaths worldwide and the third most common cause of death from all causes. In 2010, in the US alone, 222,520 new cases of lung cancer were diagnosed and 157,300 people died from this disease Approximately 85-90% of all cases of lung cancer are non-small cell lung cancer (NSCLC) (Cataldo et al. (2011) N Engl. J. Med. 364(10):947-55). Until recently, NSCLC was treated as a single disease despite recognition of its molecular and histological heterogeneity. NSCLC includes adenocarcinoma (ADC), squamous cell carcinoma, and large cell carcinoma; with recent reports indicating ADC to account for up to 50% of lung cancers. Efficacy and safety results from recent clinical trials have shown the importance of un-grouping NSCLC into its subtypes to achieve maximum benefit while minimising toxicity for patients as, unfortunately, “one size treatment does not fit all”. In light of this, there is merit in considering subtype when seeking to identify biomarkers.

Despite the devastating problem of NSCLC and the estimated 51% increased numbers of cases of this disease since 1985, a panel of reliable serum biomarkers has not yet been identified. Existing lung cancer protein biomarkers include tumour-liberated proteins such as CEA, NSE, TPA, Chromogranin, CA125, CA19-9, and Cyfra 21-1. While these are the best options currently available in the clinic, they each have limitations as detailed by Tarro et al. (2004).

The interest in circulating RNAs as biomarkers is rapidly increasing as their potential is being realised. Three years ago, we published the first whole genome microarray analysis indicating that many hundred messenger RNAs can be detected in serum (O'Driscoll et al. (2008) Cancer Genomics Proteomics. 5(2):94-104). More recently, ourselves and others have published data supporting a role for circulating miRNAs in a range of cancer types including breast (Friel et al. (2010) Breast Cancer Res Treat. 123(3):613-25; Zhao et al. (2010) PLoS One. 5(10):e13735.MCG20605), prostate (Mitchell et al. (2008) Proc Natl Acad Sci USA. 105(30):10513-8; Mahn et al. (2011) Urology. 77(5):1265.e9-1265.e16), liver (Li et al. (2011) Biochem Biophys Res Commun. 406(1):70-3; Qu et al. (2011) J Clin Gastroenterol. 45(4):355-60), gastric (Liu et al. (2010) Eur J Cancer. 47(5):784-91) and brain (Skog et al. (2008) Nat Cell Biol. 10(12):1470-6) cancers. Furthermore, a number of recent studies of NSCLC specimens collectively have substantially supported the relevance of circulating miRNAs in NSCLC (Chen et al. (2008) Cell Res. 18(10):997-1006; Hu et al. (2010) J Clin Oncol. 28(10):1721-6; Chen et al. (2011) Int J Cancer; Heegaard et al. (2011) Int. J. Cancer, Shen et al. (2011) Lab Invest. 91(4):579-87, Roth et al. (2011) Mol Oncol). Advancing on our earlier work, and supported by the important data reported in the NSCLC serum studies reported by others, here we present what we believe to be the largest global analysis of miRNAs (667 miRNAs) in serum specifically focusing on the most common type of NSCLC, adenocarcinoma.

The present inventors have surprisingly found a group of miRNAs that can be used in the diagnosis of lung adenocarcinoma.

### OBJECT OF THE INVENTION

A first object of the invention is to provide novel biomarkers for the detection of lung adenocarcinoma. The ideal biomarker should be one that can be sampled minimally invasively, and sensitively enough to detect early presence of tumors in almost all patients and absent or minimal in healthy tumor free individuals.

### SUMMARY OF THE INVENTION

According to the present invention there is provided a diagnostic kit to detect lung adenocarcinoma, or to stratify patients according to expected prognosis comprising at least one oligonucleotide probe capable of binding to at least a portion of a circulating miRNA selected from the group comprising miR-556, -550, -939, -616*, -146b-3p and -30c-1*.

The diagnostic kit may comprise at least one oligonucleotide probe capable of binding to at least a portion of a circulating miRNA selected from the group comprising miR-556, -550, -939, -616*, -146b-3p, -30c-1*, -339-5p and -656.

The kit may be adapted for performance of an assay selected from a real-time PCR assay, a micro-array assay, a histochemical assay or an immunological assay. For LRG assays cytochrome C may be used as a capturing ligand for building an ELISA. All such assays are well known to those of skill in the art. Where the assay is a histochemical assay, the antibody may be labelled with a suitable label. Suitable labels include coloured labels, fluorescent labels and radioactive labels.

The kit is capable of detecting lung adenocarcinoma, even in its earliest stage. This information is then used to guide further treatment regimens. Current methods of diagnosis and stratification of lung cancers are far from perfect, so the miRNA blood test of the invention has the potential to improve the current system and be more accurate and specific in determining the patient's treatment regimen

This novel diagnostic kit has potential for the following clinical applications:

The kit of the invention provides for the fast and accurate diagnosis of ADC. This is advantageous as it, allows the identification of the stage of ADC disease affecting a patient. As the necessary degree of treatment depends on the stage of the disease in the subject, the kit of the invention allows for a timely determination to be made by the clinician of the necessary treatment that will best address the needs of the patient. As ADC develops in stages, the faster the stage is determined, the quicker the patient may receive the necessary treatment, which will result in an overall better prognosis.

The miRNAs identified and incorporated into this kit may also serve as novel therapeutic targets for lung adenocarcinoma. The invention further provides a method of identifying a therapeutic agent capable of preventing or treating lung adenocarcinoma, comprising testing the ability of the potential therapeutic agent to alter the expression of at least one circulating miRNA selected from the group comprising miR-556, -550, -939, -616*, -146b-3p and -30c-1*. The invention may further comprise testing the ability of the potential therapeutic agent to alter the expression of at least one circulating miRNA selected from the group comprising miR-556, -550, -939, -616*, -146b-3p, -30c-1*, -339-5p and -656. By “alter”, it is meant that expression is increased or that expression is decreased.

In another aspect the invention provides use of a circulating miRNA selected from the group comprising miR-556, -550, -939, -616*, -146b-3p and -30c-1* to detect lung adenocarcinoma, or to stratify patients according to expected prognosis. In a further aspect, the use may comprise use selected from a group comprising miR-556, -550, -939, -616*, -146b-3p, -30c-1*, -339-5p and -656 to detect lung adenocarcinoma, or to stratify patients according to expected prognosis.

The detection may be carried out on a blood sample or a sample derived from blood.

The kit may be adapted for performance of an assay selected from a real-time PCR assay, a micro-array assay, a histochemical assay or an immunological assay. For LRG assays cytochrome C may be used as a capturing ligand for building an ELISA. All such assays are well known to those of skill in the art. Where the assay is a histochemical assay, the antibody may be labelled with a suitable label. Suitable labels include coloured labels, fluorescent labels and radioactive labels.

The invention also provides a method of detecting or screening for lung adenocarcinoma, comprising analysing a sample of blood taken from a patient to determine a level in the sample of one or more circulating miRNAs selected from the group comprising miR-556, -550, -939, -616*, -146b-3p and -30c-1*, the level of at least one of the miRNAs in the sample indicating the presence of lung adenocarcinoma. In an embodiment, when the level of the at least one miRNA in the sample falls above a predetermined threshold value for that miRNA, this indicates the presence of lung adenocarcinoma. Each miRNA in the group may have an independently predetermined threshold value. The threshold value for at least one of the miRNAs may be zero. The method may further comprise determining a level of a circulating miRNA in a sample selected from the group of miRNAs wherein the group further comprises miR-339-5p and miR-656. In an embodiment, when the level of the at least one miRNA selected from a subgroup comprising miR-339-5p and miR-656 falls below a predetermined threshold value, this indicates the presence of lung adenocarcinoma. The threshold value for at least one of the miRNAs selected from the group of eight miRNAs may be calculated based on an analysis of the level of at least one miRNA in one or more non-cancerous control samples.

The kits, assays and methods of the invention may comprise determining the level of at least 2 circulating miRNAs from the group, or at least 3 circulating miRNAs, or at least 4 circulating miRNAs, or at least 5 circulating miRNAs, or at least 6 circulating miRNAs, or at least 7 circulating miRNAs, or at least 8 circulating miRNAs from the group. In methods of the invention where the levels of at least 2 or more circulating miRNAs are determined and compared to a threshold, each miRNA may be compared to a separate, dedicated threshold. Alternatively, in methods of the invention where the levels of at least 2 or more circulating miRNAs are determined and compared to a threshold, the levels of the at least 2 circulating miRNAs may be expressed as a function and compared to a single compound threshold.

“Synthetic oligonucleotide” refers to molecules of nucleic acid polymers of 2 or more nucleotide bases that are not derived directly from genomic DNA or live organisms. The term synthetic oligonucleotide is intended to encompass DNA, RNA, and DNA/RNA hybrid molecules that have been manufactured chemically, or synthesized enzymatically in vitro.

An “oligonucleotide” is a nucleotide polymer having two or more nucleotide subunits covalently joined together. Oligonucleotides are generally about 10 to about 100 nucleotides. The sugar groups of the nucleotide subunits may be ribose, deoxyribose, or modified derivatives thereof such as OMe. The nucleotide subunits may be joined by linkages such as phosphodiester linkages, modified linkages or by non-nucleotide moieties that do not prevent hybridization of the oligonucleotide to its complementary target nucleotide sequence. Modified linkages include those in which a standard phosphodiester linkage is replaced with a different linkage, such as a phosphorothioate linkage, a methylphosphonate linkage, or a neutral peptide linkage. Nitrogenous base analogs also may be components of oligonucleotides in accordance with the invention.

A “target nucleic acid” is a nucleic acid comprising a target nucleic acid sequence. A “target nucleic acid sequence,” “target nucleotide sequence” or “target sequence” is a specific deoxyribonucleotide or ribonucleotide sequence that can be hybridized to a complementary oligonucleotide.

An “oligonucleotide probe” is an oligonucleotide having a nucleotide sequence sufficiently complementary to its target nucleic acid sequence to be able to form a detectable hybrid probe:target duplex under high stringency hybridization conditions. An oligonucleotide probe is an isolated chemical species and may include additional nucleotides outside of the targeted region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labelled with a detectable moiety such as a radioisotope, a fluorescent moiety, a chemiluminescent, a nanoparticle moiety, an enzyme or a ligand, which can be used to detect or confirm probe hybridization to its target sequence. Oligonucleotide probes are preferred to be in the size range of from about 10 to about 100 nucleotides in length, although it is possible for probes to be as much as and above about 500 nucleotides in length, or below 10 nucleotides in length.

A “hybrid” or a “duplex” is a complex formed between two single-stranded nucleic acid sequences by Watson-Crick base pairings or non-canonical base pairings between the complementary bases. “Hybridization” is the process by which two complementary strands of nucleic acid combine to form a double-stranded structure (“hybrid” or “duplex”).

“Complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or uracil (U), while guanine (G) ordinarily complements cytosine (C).

The term “stringency” is used to describe the temperature, ionic strength and solvent composition existing during hybridization and the subsequent processing steps. Those skilled in the art will recognize that “stringency” conditions may be altered by varying those parameters either individually or together. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. Stringency conditions are chosen to maximize the difference in stability between the hybrid formed with the target and the non-target nucleic acid. This is well within the ability of one skilled in this art.

With “high stringency” conditions, nucleic acid base pairing will occur only between nucleic acid fragments that have a high frequency of complementary base sequences (for example, hybridization under “high stringency” conditions, may occur between homologs with about 85-100% identity, preferably about 70-100% identity). With medium stringency conditions, nucleic acid base pairing will occur between nucleic acids with an intermediate frequency of complementary base sequences (for example, hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

‘High stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, ph adjusted to 7.4 with NaOH), 0.5% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 0.1×SSPE, 1.0% SDS at 42° C. when a probe of about 500 nucleotides in length is used. “Medium stringency’conditions are those equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 10.0×SSPE, 1.0% SDS at 42° C., when a probe of about 500 nucleotides in length is used.

‘Low stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent and 100 μg/ml denatured salmon sperm DNA followed by washing in a solution comprising 5×SSPE, 0.1% SDS at 42° C. when a probe of about 500 nucleotides in length is used.

The examples above are for probes of about 500 nucleotides in length. However, it is well known in the art that the use of probes of smaller lengths, such as miRNAs, requires an increase in the stringency conditions, see protocol of Varallay et al, Nature Protocols, Vol. 3 No. 2 (2008). The way in which the stringency is increased is well known in the art and can achieved by altering the ‘washing’ step by way of decreasing the salt concentration via a decrease in the concentration of SSPE buffer and/or increasing the % of SDS and/or increasing the temperature.

In the context of nucleic acid in-vitro amplification based technologies, “stringency” is achieved by applying temperature conditions and ionic buffer conditions that are particular to that in-vitro amplification technology. For example, in the context of PCR and real-time PCR, “stringency” is achieved by applying specific temperatures and ionic buffer strength for hybridisation of the oligonucleotide primers and, with regards to real-time PCR hybridisation of the probe/s, to the target nucleic acid for in-vitro amplification of the target nucleic acid.

One skilled in the art will understand that substantially corresponding probes of the invention can vary from the referred-to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from about 100% to about 80% or from 0 base mismatches in about 10 nucleotide target sequence to about 2 bases mismatched in an about 10 nucleotide target sequence. In preferred embodiments, the percentage is from about 100% to about 85%. In more preferred embodiments, this percentage is from about 90% to about 100%; in other preferred embodiments, this percentage is from about 95% to about 100% e.g., 95, 96, 97, 98, 99, or 100%.

The terms “identical” or percent “identity,” in the context of two or more nucleic acids or polypeptide sequences, refer to two or more sequences or subsequences that are the same or have a specified percentage of amino acid residues or nucleotides that are the same (i.e., 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman (1981) Adv. Appl. Math. 2:482, by the homology alignment algorithm of Needleman & Wunsch (1970) J. Mol. Biol. 48:443, by the search for similarity method of Pearson & Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

By “sufficiently complementary” or “substantially complementary” is meant nucleic acids having a sufficient amount of contiguous complementary nucleotides to form, under high stringency hybridization conditions, a hybrid that is stable for detection.

By “nucleic acid hybrid” or “oligonucleotide:target duplex” is meant a structure that is a double-stranded, hydrogen-bonded structure, preferably about 10 to about 100 nucleotides in length, more preferably 14 to 50 nucleotides in length, although this will depend to an extent on the overall length of the oligonucleotide probe. The structure is sufficiently stable to be detected by means such as chemiluminescent or fluorescent light detection, autoradiography, electrochemical analysis or gel electrophoresis. Such hybrids include RNA:RNA, RNA:DNA, or DNA:DNA duplex molecules.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

By “preferentially hybridize” is meant that under high stringency hybridization conditions oligonucleotide probes can hybridize their target nucleic acids to form stable probe:target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid to enable one skilled in the art to accurately detect the presence of (for example Candida) and distinguish these species from other organisms. Preferential hybridization can be measured using techniques known in the art and described herein.

### BRIEF DESCRIPTION OF THE DRAWINGS

FIG. 1. miR-556, -550, -939, -616*, -146b-3p and -30c-1* were detected at substantially higher amounts in serum from ADC patients (n=40) compared to their individually (A) or mean value (B) for their paired age- and gender-matched controls (n=40). miR-339-5p and miR-656 were detected at substantially lower levels in serum from ADC patients (n=40), as shown after comparing their individual (C) or mean value (D) for their paired age- and gender-matched controls (n=40). Graphs represent fold increase in ADC (mean+/−SE)

FIG. 2. Considering ADC tumour Stage, miR-556, -550, -939, -616*, -146b-3p and -30c-1* were detected at substantially higher amounts in serum from Stage 1 ADC patients (n=10) compared to their individually paired age- and gender-matched controls. The circulating amounts of each of these miRNAs increased significantly again in Stage 2 compared to Stage 1, before decreasing again in Stage 3 disease and then increasing again, to some extent, in Stage 4. Conversely, miR-339-5p levels were decreasing from Stage 1 to Stage 2 and then to Stage 3 with a lesser effect from Stage 3 to Stage 4; although this was not significant. A similar trend was observed for miR-656 except that the reduced levels in Stages 1 and 2 disease did not differ significantly from each other. Graphs represent fold increase in ADC compared to their individually paired age- and gender-matched controls (mean+/−SE).

FIG. 3. Considering ADC tumour Stage, miR-556, -550, -939, -616*, -146b-3p and -30c-1* were detected at substantially higher amounts whereas, miR-339-5p and miR-656 was down-regulated in serum from all Stages of ADC patients compared to the mean detection level in the paired age- and gender-matched controls; although a direct association was not found with disease stage. Graphs represent fold increase in ADC compared to their individually paired age- and gender-matched controls (mean+/−SE).

FIG. 4. Co-analysis of miR-556, -550, -939, -616*, -146b-3p and -30c-1* shows significantly increased levels in ADC sera overall compared to their collective levels in paired age- and gender-matched controls. Increased levels of these 6 miRNAs were found in Stage 2 sera compared to that in Stage 1, but fell again in Stage 3 before rising in Stage 4 (A). Co-analysis of miR-339-5p and miR-656 showed reduced levels in ADC sera overall compared to their combined levels in paired age- and gender-matched controls (B). Graphs represent fold increase in ADC compared to the mean levels in control sera (mean+/−SE).

### DETAILED DESCRIPTION OF THE PREFERRED EMBODIMENTS

The aim here was to apply global profiling approaches to explore miRNAs in serum from patients and with ADC of the lung, investigating if these miRNAs may have potential as diagnostic biomarkers. This study involved RNA isolation from 80 sera specimens including those from patients with ADC (equal numbers of Stages 1, 2, 3 and 4) and age- and gender-matched controls (n=40 each). 667 miRNAs were co-analysed in these specimens using TaqMan low density arrays. Individual miRNAs were selected for qPCR validation. Successful isolation of RNA was achieved from all sera specimens. The quantities of RNA in ADC and control sera did not differ significantly (p=0.470). Overall, approximately 390 and 370 miRNAs, respectively, were detected in ADC and control sera. A group of six miRNAs, miR-30c-11*, miR-616*, miR-146b-3p, miR-566, miR-550 and miR-939, was found to be present at substantially higher levels in ADC compared to control sera. Conversely, two further miRNAs. miR-339-5p and miR-656 were detected at substantially lower levels in the serum from ADC patients compared to control sera. Furthermore, co-analysis of these miRNAs showed a correlation between miRNA expression and progression from Stages 1 to Stage 2 disease; although the numbers of specimens included was too limited to derive a meaningful statistical relevance on. Differences in miRNA profile identified here suggest that circulating miRNAs may have potential as diagnostic biomarkers for ADC. Of particular interest, we believe that this panel of six miRNAs has never previously been associated with serum or with ADC.

### Material and Methods

### Patient Characteristics

The study involved the analysis of 667 miRNAs in 80 serum specimens. Forty-two of the specimens were procured from consenting patients who were diagnosed with adenocarcinoma (ADC) of the lung. Serum specimens from 40 age-, gender- and BMI-matched healthy volunteers were analysed as controls.

### RNA Extraction

RNA was isolated from 250 μl of each 0.45 μm-filtered serum specimen by extracting with TriReagent (Sigma; Poole, England) using a modification of the procedure that we previously reported (O'Driscoll et al. (2008) Cancer Genomics Proteomics. 5(2):94-104). RNA was subsequently assessed at 230 nm, 260 nm and 280 nm using a Nanodrop ND-1000 (Labtech International, Ringmer East Sussex).

Global Analysis of miRNAs

Global profiling of miR expression was performed using the TaqMan Array Human Microarray Panel, representing 667 miRNAs on 2 array acrd/specimen analysed i.e. TaqMan Low Density Array (TLDA) panel A (377 miRNAs) and panel B (290 miRNAs) (Applied Biosystems, CA, USA). cDNA was prepared from three μl RNA (25 ng/μl) according to the ABI microRNA TLDA Reverse Transcription Reaction protocol. The cDNA product (2.5 μl per specimen) was pre-amplified according to the ABI TLDA pre-amplification protocol. The ABI Taqman microRNA low density arrays (TLDA, Applied Biosystems) were selected as the platform for microRNA profiling. The amplified product was then quantified using an Applied Biosystems 7900 HT Real-Time PCR system. For initial screening, pooled specimens (equal quantities) of RNA for each cancer Stage versus pooled specimens of each set of matched controls were evaluated. Subsequent to the success of this step, individual specimens were analysed.

Real-Time Quantification of micro-RNAs

Validation of miRNAs by single and co-analysis was performed using qRT-PCR analysis (Applied Biosystems TaqMan® Micro-RNA Assay). This assay includes a reverse transcription (RT) step using the TaqMan microRNA reverse transcription kit (Applied Biosystems, CA, USA), reverse-transcribed with a MultiScribe reverse transcriptase. Briefly, the RT reaction consisted of 1.5 μL 10×RT Buffer, 0.15 μL dNTPs 100 mM, 0.19 μL RNase Inhibitor 20 U/μL, 1.0 MultiScribe reverse transcriptase, 3 μL of primer and 5 ng total RNA in a final volume of 15 μL. The reaction was then incubated in using a 7900 HT Real-Time PCR system for 30 min at 16° C., 30 min at 42° C., 5 min at 85° C., and then held at 4° C. The RT products were subsequently amplified with sequence-specific primers using the Applied Biosystems 7900HT Real-Time PCR system. The 20 μL PCR mix contains 1.33 μL RT product, 1 μL TaqMan® Universal PCR Master Mix (20×), 1 μL TaqMan® probe. The reactions were incubated in a 96-well plate at 95° C. for 10 min followed by 40 cycles of 95° C. for 15 mins and at 60° C. for 1 min.

### Data Analysis

The ABI TaqMan SDS v2.3 software was utilized to obtain raw C_{T} values. As each TLDA was performed for a given specimen (n=80) based on fixed, constant quantities of RNA in each case, to avoid introducing any bias at this stage, the raw C_{T} data (SDS file format) were exported from the Plate Centric View. P-values (T-test; significance=<0.05) and fold change was calculated using the following 2-ΔC_{T} described by Livak and Schmittgen, 2001). For analysis of TLDA data, values for each specimen were normalised to the mean of the C_{T} values. Fold changes in ADC serum versus control serum were thus determined by the ΔC_{T} method as described previously i.e. cycle threshold (C_{T}) ADC-(C_{T}) control (Livak and Schmittgen (2001) Methods 25:402-408; Chen et al. (2008) Cell Res. 18(10):997-1006; Hu et al. (2010) J Clin Oncol. 28(10):1721-6; Hennessey et al. (2012)). Excel and SPSS 16.1 statistics packages were used. To assess sensitivity and specificity, receiver operating characteristic (ROC) curves were created using GraphPad.

### Results

### Patient Characteristics

This study involved analysis of 80 serum specimens, including 40 sera from patients (22 male and 18 female) with ADC and from 40 age-, gender- and BMI-matched healthy volunteers. Regarding cigarette smoking history for the patient cohort, 29% never smoked, 16% previously smoked, and 55% were smokers at the time of diagnosis. Forty-eight percent of the controls never smoked, 30% previously smoked and 22% are current smokers. There was no significant (p=0.470) difference between the ages of the individual included in each group i.e. ADC patients had a median and mean age of 65 yrs. For controls, the median and mean age was 64 years. Table 1 summarises the gender balance and age following sub-division of the matched specimens based on ADC Stage at which the patients presented.

TABLE 1
Adenocarcinoma patients and healthy controls

| | Controls | ADC | |
|---|---|---|---|
| miRNA | (Yrs; mean +/− SD) | (Yrs; mean +/− SD) | P value |
| Stage 1 | 59.9 +/− 2.0 | 62.1 +/− 2.0 | 0.450 |
| (6 male; 4 female) | | | |
| Stage 2 | 67.6 +/− 40 | 68.9 +/− 3.5 | 0.810 |
| (6 male; 4 female) | | | |
| Stage 3 | 58.2 +/− 2.4 | 60.2 +/− 3.2 | 0.630 |
| (5 male; 5 female) | | | |
| Stage 4 | 70.9 +/− 2.0 | 70.0 +/− 2.7 | 0.790 |
| (5 male; 5 female) | | | |

RNA Yield and miRNA Presence

Total RNA quantification from each serum specimen showed the yields to be similar from the patient and control cohort. Specifically for each 250 μl of patients serum, an average of 1.88+/−0.33 μg RNA was retrieved, with control sera producing a mean of 1.83+/−0.2 μg RNA (p=0.98).

The results from this study of 667 miRNAs evaluated by low density arrays showed that the numbers of miRNAs present in ADC and control sera do not differ substantially. Assuming C_{T} values of <35 as indicative of miRNA presence, 230+/−51 miRNAs were detected in serum from ADC patients and 240+/−21 were detected in control sera (p=0.729). Applying less stringent C_{T} values of <40 as present, 326+/−68 miRNAs were detected in patients sera and 336+/−36 in control sera (p=0.759).

Assessing for miRNAs Reported to Generally be Present in Serum or Plasma

A number of miRNAs have been reported as typically present in serum/plasma including miR-16, miR-103, miR-93, miR-192 and miR-451. As expected, we found these miRNAs to be present in all specimens analysed, with no significant differences in detection level between the 40 sera specimens from ADC patients and the 40 normal sera (see Table 2).

TABLE 2
Assessment of 5 miRNAs commonly detected in serum or plasma

| miRNA | Control (Mean C_{T}) | ADC (Mean C_{T}) | P value |
|---|---|---|---|
| miR-16 | 21.5 +/− 1.5 | 22.2 +/− 2.9 | 0.748 |
| miR-103 | 28.7 +/− 1.6 | 30.1 +/− 2.2 | 0.351 |
| miR-93 | 26.5 +/− 1.1 | 27.7 +/− 3.1 | 0.604 |
| miR-192 | 29.5 +/− 0.9 | 29.9 +/− 2.5 | 0.772 |
| miR-451 | 25.0 +/− 1.8 | 26.4 +/− 4.2 | 0.640 |

miRNAs Identified as Associated with ADC Using Taqman Low Density Arrays

TaqMan low density arrays showed 3 miRNAs to be undetectable (assuming no amplification by 40 C_{T} to indicated absence) in all 40 control sera specimens, and present in ADC sera at all stages of disease. These are miR-556, miR-550 and miR-939. A number of other miRNAs, while present at low levels in some control sera, were found to be present at substantially higher levels in ADC sera compared to control. Specifically, the mean fold increases for these miRNAs in ADC serum specimens compared to control sera were as follows: miR-517c, 12.3 fold (range: 2.3-18.8 fold); miR-770-5p, 17.3 fold (range: 2.1-40.3); miR-605, 26.7 fold (range 2.1-42.0 fold); miR-212, 9 fold (range: 4.1-23.0 fold); miR-601, 6.8 fold (range: 3.3-14.6 fold). When all data was normalised to mean C_{T}, prior to comparison of ADC C_{T} to control C_{T} values, the mean fold increases for these miRNAs in ADC serum specimens compared to control sera were as follows: miR-517c (21.6 fold; range: 2.1-63.9 fold); miR-770-5p (15.8 fold; range: 2.0-36.6); miR-605 (50.4 fold; range 1.2-143.3 fold); miR-212 (10.7 fold; range: 2.3-21.6 fold); miR-601 (7.8 fold; range: 3.1-13.2 fold). Conversely, two miRNAs were found to be at substantial higher levels across the 40 normal sera specimens compared to ADC sera i.e. miR-656 and miR-339 were detected at, on average, 20.1-fold (range: 2.6-37.4 fold) and 22.7-fold (range: 3.3-62.7 fold) higher levels in control compared to ADC serum specimens. When this data was normalised to mean C_{T}, prior to comparison of ADC C_{T} to control C_{T} values, the mean fold increases were as follows: miR-656 (22.8-fold; range: 2.8-44.5 fold) and miR-339-5p (21.4-fold; range: 4.8-69.1 fold).

qPCR Validation of Results Arising from TLDA Analysis

Array technology enabled co-analysis of many (667) miRNAs. However, in order to establish if the results from such analysis would consistently be found using a more routine technique that could potentially be translated to hospital laboratories for analysis, 6 initial miRNAs and two subsequent miRNAs were selected for individual analysis in all 80 specimens using standard quantitative polymerase chain reaction (qPCR) analysis. This more limited group of miRNA was selected as RNA quantities available were limited. However, these would prove in principle if validation would be achieved. The fact that little, if any, information is published on these miRNAs means that their selection also adds to the advancement of our understanding of miRNAs. Specifically, these miRNAs included miR-556, miR-550 and miR-939 (found by TLDAs to be absent from control sera (n=40) and present in ADC sera (n=40)). The other 3 miRNAs selected for qPCR analysis were miR-616*, miR-146b-3p and miR-30c-1* which were identified as potential biomarkers for ADC in a more limited pilot study of Stage 1 ADC only (n=10) and age- and gender-matched control (n=10) sera in accordance with the section entitled “Supplementary Material” below. The fact that this trend was also found through the TLDA analysis here i.e. miR-616*, miR-146b-3p and miR-30c-1* were present (≦35 C_{T}) in the Stage 1, but were absent from matched control sera supported their further investigation. The other two miRNAs selected for assessment by qPCR were miR-339-5p and miR-656, that were identified as at substantially lower levels in ADC sera compared to control specimens.

miR-566:

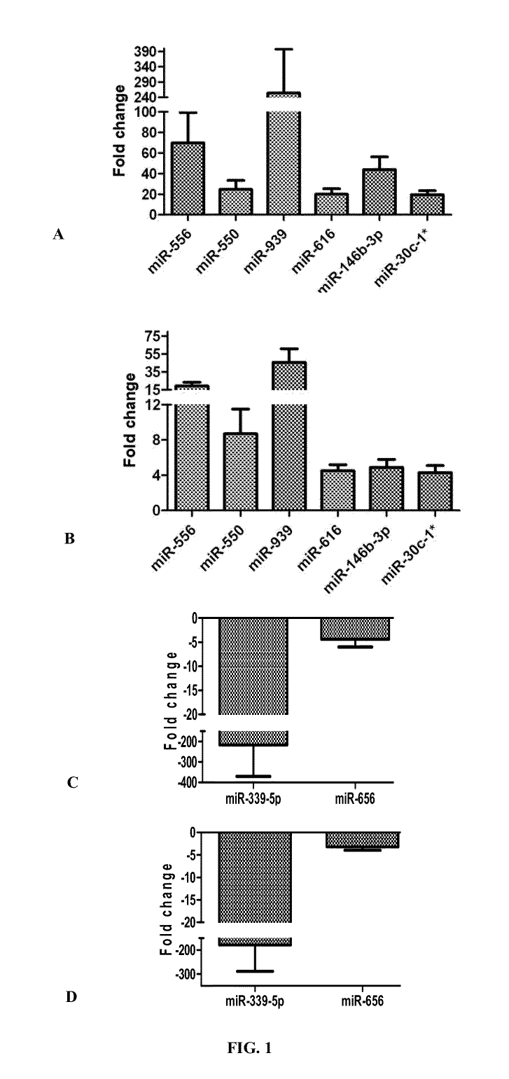

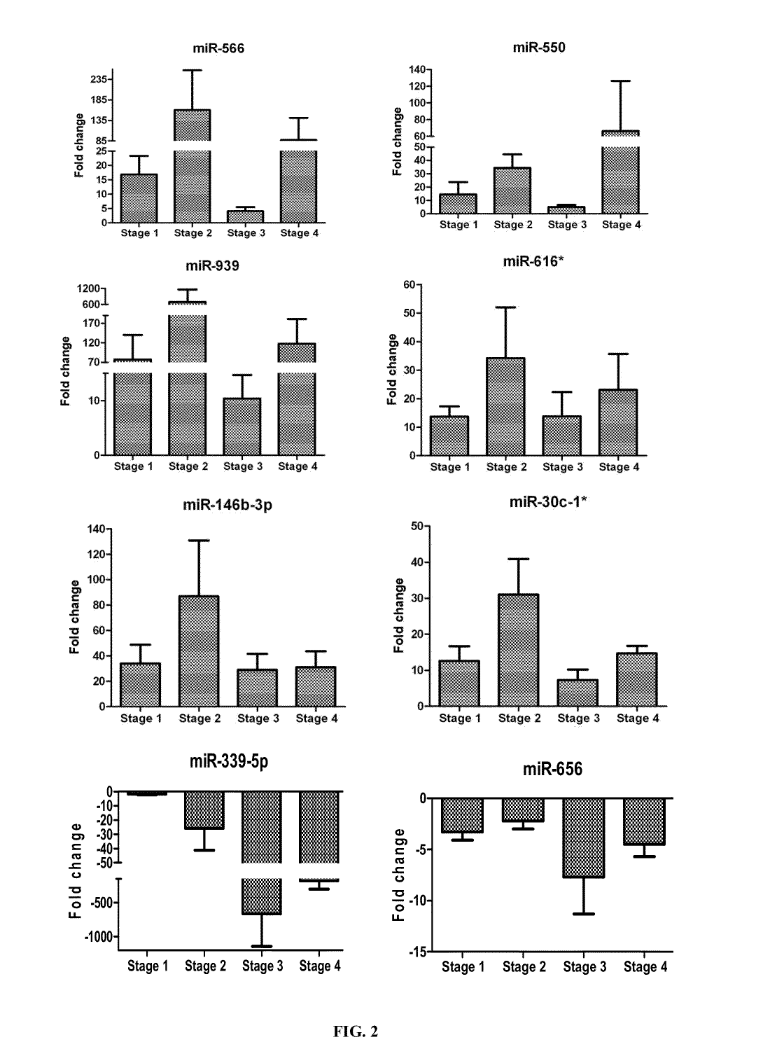

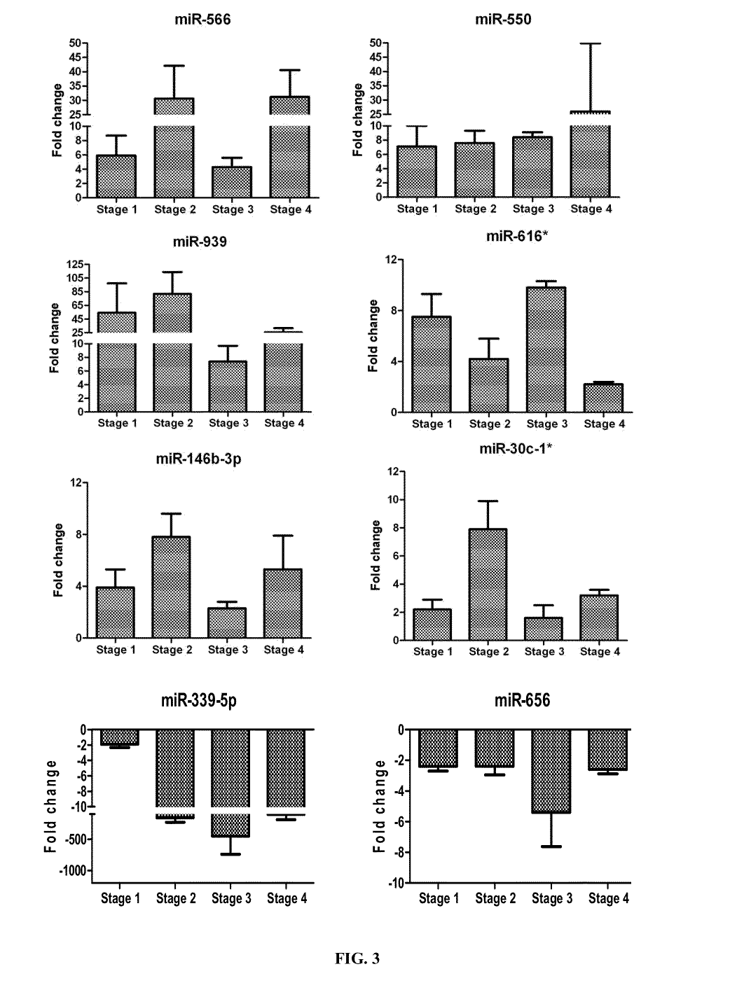

Using quantitative PCR analysis, miR-566 was detected in all specimens with the exception of one ADC specimen. Directly comparing each ADC and matched control showed miR-556 to be 70+/−29.4 fold increased in ADC sera, in all but 5 matched pairs (FIG. 1(A)). As individual matched normal specimens would not necessarily always be available for comparison, we also analysed levels in each ADC specimen compared to the overall mean levels in the 40 controls; showing a 19.1+/−4.4 fold increase in 95% of cases (see FIG. 1(B)). Considering the 4 Stages of ADC, levels of circulating serum miR-556 in ADC specimens (compared to their individual matched control pairs) were found to increase in Stage 2 disease versus to Stage 1. However, levels in Stage 3 decreased substantially compared to Stage 2 before increasing again in Stage 4 disease (see FIG. 2). This trend was also observed when miR-556 in individual ADC sera were compared to the mean level in control specimens (see FIG. 3).

miR-550:

miR-550 was detected in 100% of ADC sera. In 15% of comparison pairs (6/40) miR-550 went from undetectable in normal serum to present in ADC. While some level of miR-550 was detectable in 34 of the normal sera, the amounts were substantially greater in ADC compared to control sera in the majority (75%) of cases; with an average fold increase of miR-550 in ADC sera of 24.6+/−8.8 (FIG. 1(A)). when compared to its matched control or 8.7+/−2.8 when compared to the mean of the controls (FIG. 1(B)). For miR-550, the AUC value from ROC analysis was 0.72, showing a significant (p=0.0006) difference between ADC patients and healthy controls. When considering age- and gender-matched pair comparisons, serum levels of miR-550 increased in Stage 2 disease compared to Stage 1, with levels in Stage 3 decreasing substantially compared to Stages 1 and 2, before increasing again in Stage 4 disease (FIG. 2). Comparison of each ADC with the mean of control values indicated a marginal increase from Stage 1 to Stage 2 to Stage 3, with an apparently more substantial increase at Stage 4 (FIG. 3). However, it should be noted that this increase is strongly influenced by one Stage 4 ADC serum specimen that had exceptionally high levels of miR-550. Eliminating this specimen bring the average fold increase in Stage 4 to a similar level to that in Stage 1-3 inclusively.

miR-939: miR-939 was detected in 100% of serum specimens and was found to be at substantially higher level (i.e. 254.2+/−143.4 fold) in 85% of cases where ADC specimens were compared directly to their age- and gender-matched control sera (FIG. 1(A)). Comparison of each ADC specimen to the mean level of miR-939 in control sera showed an average increase in ADC of 45.6+/−15.2 fold (FIG. 1 (B)). Of note, the same levels of miR-939 were detected in one ADC specimen when compared to its matched control levels, while 3 (Stage 3) sera specimens had slightly lower levels of miR-939 compared to control, reflecting a mean difference of (1.7+/−0.5 C_{T}). Considering the 4 disease Stages, both matched-pair comparisons and comparisons of individual ADC specimen levels with the mean control level showed levels of circulating serum miR-939 increased in Stage 2, with levels in Stage 3 decreasing substantially compared to Stages 1 and 2, before increasing again in Stage 4 disease (see FIGS. 2 & 3).

miR-616*:

miR-616* was detected in 98% of ADC serum specimens. In 30% of matched specimens, miR-616* went from undetectable in controls to being present in ADC. While miR-616* was within detectable levels in 27 of control sera, in the majority (82.5%) of matched specimens, the amounts were substantially higher level (i.e. 20+/−5.2 fold) in ADC compared to individual paired control sera (FIG. 1(A)) The miR-616* AUC value from ROC analysis was 0.71, demonstrating a significant (p=0.001) difference between ADC patients and healthy controls. The increased levels of miR-616* in ADC compared to mean of controls was found to be 4.5+/−0.7 fold (FIG. 1(B)). Levels of miR-616* detectable in ADC serum did not consistently correlate with disease Stage (see FIGS. 2 & 3).

miR-146b-3p:

miR-146b-3p was detected in 95% of ADC serum specimens. In 51.5% of matched specimens compared it went from undetectable in controls to being present in ADC. In 5% of cases this miRNA was absent from both the ADC and its matched control specimen. Where miR-146b-3p was detected in both ADC and control sera, the general trend was substantially higher levels (i.e. 44+/−12.3 fold) in ADC compared to age- and gender-matched control sera (FIG. 1(A)). For miR-146b-3p, the AUC value from ROC analysis was 0.82; demonstrating a significant (p<0.0001) difference between ADC patients and healthy controls. This was reflected as 4.9+/−0.9 fold when comparing individuals ADC specimens that showed increased levels of miR-146b-3p to the average levels in the controls (FIG. 1(B)). Considering the 4 Stages of ADC, as for miR-556, levels of circulating miR-146b-3p increased in Stage 2 disease compared to Stage 1. However, levels in Stage 3 & 4 decreased compared to Stage 2 (see FIGS. 2 & 3).

miR-30c-1*:

miR-30c-11* was detected, by qPCR, in 70% of ADC serum specimens and in 28% of control sera. In 53% of cases, miR-30c-1* went from undetectable in controls to being present in ADC. When miR-30c-1* were detected in control serum, in general the amounts present were substantially higher (i.e. 19.5+/−3.9 fold) in early stage ADC compared to their respective matched controls. Of note, in a limited number of matched pairs (15%; 6/40) lower levels of miR-30c-1* were found in ADC compared to matched control sera. Overall, however, the AUC value from miR-30c-1* ROC analysis was 0.74 demonstrating a significant (p=0.00018) difference between ADC patients and healthy controls. Comparing increased levels of miR-30c-1* in each ADC sera specimen, a mean increase of 4.3+/−0.8 was found, compared to the average in controls. Again a minority (12.5%) of ADC specimens showed lower levels (2.1+/−0.5) of this miRNA compared to matched controls in early disease. Considering the 4 disease Stages, as for a number of other miRNAs evaluated, miR-30c-1* levels increase in Stage 2 disease compared to Stage 1, with levels in Stage 3 decreasing compared to Stage 2, before increasing again in Stage 4 disease (see FIGS. 2 & 3). Importantly, while miR-30c-1* was detectable in only 70% of ADC specimens overall, its absence was restricted to the earlier stages of the diseases and, importantly, miR-30c-1* was detected in 100% of Stage 4 specimens.

miR-339-5p:

qPCR analysis confirmed that the levels of miR-339-5p were substantially lower in serum from ADC patients compared to that from healthy controls (FIG. 1(C)). Considering the individual stages of disease, miR-339-5p was substantially lower in 40% and 70% of the Stage 1 and Stage 2, respectively, and in 100% of both Stage 3 and Stage 4 ADC serum specimens. The AUC value from miR-339-5p ROC analysis was determined to be 0.6.

miR-656:

qPCR analysis also validated our TLDA analysis of miR-656 i.e. miR-656 level was down in ADC serum specimens compared to their age- and gender-matched control sera (FIG. 1(D)). This was found to be the situation in 40% of Stage 1 specimens, 60% of Stage 2 specimens, and 70% of both Stages 3 and 4. The AUC value from miR-656 ROC analysis was 0.6.

Co-Analysis of Panel of miRNAs in all Specimens

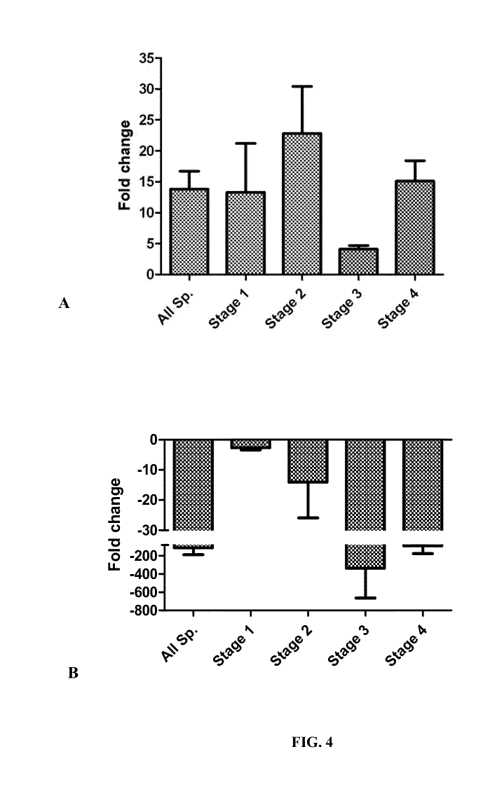

As all 6 of the initial miRNAs identified as potential panel members were not over-expressed in 100% of ADC specimens, we co-assessed their expression. A minimum of 2 miRNAs and up to the maximum of all 6 miRNAs were over-expressed in any given ADC specimen. This emphasises the relevance of assessing all 6 miRNA. Considering all 6 miRNAs, the AUC value from ROC co-analysis was 0.7, indicating a significant (p<0.0001) difference between ADC patients and healthy controls. As indicated in FIG. 4, co-analysis of the miRNAs show a 13.8+/2.9 fold increase levels in ADC compared to control sera. Considering each stage of disease individually, this was reflected in their increased levels in Stage 2 compared to Stage 1, with reduced levels in Stage 3 sera before increasing again in Stage 4. In the relation to the combination of the two subsequent miRNAs (miR-339-5p and miR-656) reduced in ADC sera, the AUC value from ROC co-analysis was 0.6, indicating a significant (p=0.02) difference between ADC patients and healthy controls. As shown in FIG. 4B, co-analysis of these two miRNAs show 110.7+77.7 fold decrease in levels in ADC compared to control sera. Considering each stage of disease individually, this was reflected in their decreased levels from Stage 1 to Stage 2 to Stage 3, with no substantial difference noted between Stage 3 and Stage 4.

### Discussion

ADC of the lung is currently the single biggest killer in cancer. Studies by us and others strongly support a potential role for RNAs as circulating minimally-invasive biomarkers. In fact, a number of recently published and emerging studies suggest that miRNAs exist in sera that are associated, in general, with non-small cell lung cancer. Advancing on this, here we report what we believe to be the first large study (677 miRNAs) of circulating miRNAs specifically in ADC. Our study compared the miRNA profile of ADC with to age- and gender-matched control sera. The main novel findings of this study include the observation that there are >300 miRNAs detectable in serum; while many (270-290) miRNAs are present in serum from healthy controls as well as ADC patients, a number of miRNAs are differentially detected (based on absent versus presence or differential levels of detection) under these circumstances. Here we identified a group of 6 miRNAs that exist at substantially higher levels in the ADC compared to control sera. We consistently found increased amounts of these miRNAs to be present in serum from patients with Stage 2 disease compared to Stage 1, with levels reduced in Stage 3 before rising again in Stage 4. In addition, we identified a group of 2 miRNAs that exist at lower levels in ADC compared to control sera.

In relation to numbers of circulating miRNAs and considering relevant studies performed by others, Chen et al. (2008) Cell Res. 18(10):997-1006 reported on an important study including analysis of serum from 7 young Chinese subjects where over 100 and 91 miRNAs, respectively, were detected in male and female subjects. Assessing cohorts of 30 NSCLC patients based on disease survival, Hu et al. (2010) J Clin Oncol. 28(10):1721-6 detected 109 miRNAs and 101 miRNAs in the serum from patients with longer- and shorter-survival times, respectively. In the study reported here which including serum from 44 males and 36 females, we did not find any association between miRNA numbers and gender. This is in agreement with a recent study by Heegaard et al. (2011) Int. J. Cancer, where no association was found between gender and serum/plasma miRNA profiles. However, compared to the study by Chen et al. (2008) Cell Res. 18(10):997-1006, we detected many more sera miRNAs overall i.e. approximately 390 and 370 miRNAs in ADC and control sera, respectively. The greater number of miRNAs detected here may be due to a combination of factors, including advancement on technology for miRNAs identification and evaluation—and so the numbers of miRNAs known to exist and detectable—as well as the somewhat larger cohorts of cases possible for us to evaluate. Of note, Heegaard et al. (2011) Int. J. Cancer reported considerable difference in miRNA levels (amounts 14 miRNAs significantly reduced in serum from African American compared to European Americans) so it is conceivable that, as with many genetic and phenotypic traits associated with cancer, race may some way contribute to circulating miRNA profiles; emphasis the importance of increasing the numbers of international collaborative studies in this field. Overall, we believe that our work complements studies by Chen et al. (2008) Cell Res. 18(10):997-1006 and J Clin Oncol. 28(10): 1721-6 and collectively adds to our understanding of the numbers and scope of miRNAs in the circulation.

In relation to disease biomarkers, assessing NSCLC overall as a single disease (Chen et al. (2011; IJC in press), evaluated 91 miRNAs and identified 10 of these as potential biomarkers for NSCLC. Importantly their study did not include analysis of the 6 miRNAs (miR-30c-1*, miR-616*, miR-146b-3p, miR-566, miR-550 and miR-939) which we detail in this study of ADC. Of the 10 miRNAs reported as differentially expressed, Chen et al. (2011), miR-199a-5p was found to be substantially (15.64 fold) increased in NSCLC compared to control sera. In keeping with this, we found miR-199a-5p to be present in ADC sera but absent from control sera. The remaining 9 miRNAs reported by Chen et al. (2011) were not substantially different in our ADC and control sera. Differences in these two observations are likely to be contributed to by the fact that our study was specifically of ADC, while no specific associations with NSCLC subtype were investigated by Chen et al. (2011).

In their study of 30 serum miRNAs, in NSCLC compared to controls, Heegaard et al. (2011) Int. J. Cancer observed reduced quantities of 7 miRNAs including miR-221, let-7a, -155, 17-5p, -27a, -106a and -146b. Interestingly our microarray analysis showed a similar trend for miR-221, let-7a, 17-5p, -27a and -106a.

For miR-155, we observed increased levels in Stage 1 disease, but reduced levels for Stages 2-4 inclusively (and so we did not consider this to be one of the most relevant miRNAs from our study). The discrepancy with miR-155 between the study by Heegaard et al. (2011) Int. J. Cancer and the work presented here may, again, be attributed to the disease being analysed (NSCLC collectively versus ADC) and the stage of disease i.e. Heegaard et al. (2011) Int. J. Cancer included Stages 1 and 2 of NSCLC, while we considered all 4 Stages of ADC). Of note, in a study of serum from 35 lung cancer patients (including 18 small cell lung cancers and 9 NSCLC— but the subtypes were not defined), Roth et al. (2011) reported levels of miR-155 to be significantly higher in lung cancer compared to benign disease.

Our data on miR-146b conflicted with that found by Heegaard et al. (2011) Int. J. Cancer i.e. miR-146b levels were substantially increased in our ADC but reduced in the NSCLC analysed by Heegaard et al. (2011) Int. J. Cancer.

Included here, are some points regarding the increased amounts of “our 6 miRNAs” in Stage 2 versus Stage 1. Our data suggests a potential association with early events of ADC development and possibly associated inflammatory events. Further studies with larger sample populations will provide additional evidence as to this observation in relation to tumour stages.

### CONCLUSION

Many observations are in agreement with more general studies of NSCLC serum or, indeed, cancer tissue, performed by others. However, through global analysis of 667 miRNAs in ADC alone we have been able to identify a group of 6 miRNAs, increased levels of which are associated with the presence of ADC. While independent validation in much larger cohorts are now warranted, we believe that that this study adds novel information to this field of circulating miRNAs and the quest to identify biomarkers for diagnosis and, ultimately, more personalized management of cancer patients.

### SUPPLEMENTARY MATERIAL

The preliminary data referred to was based on once-off exploratory assays (i.e. n=1 assays, rather than our typical n=3).

For this pilot study, serum specimens from patients with Stage 1 ADC and age-matched controls (n=10 each) were purchased from a biobank (Asterand; http://www.asterand.com). RNA was isolated these 250 μl serum specimen after passing through a 0.45 μm-filter. RNA was extracted with TriReagent (Sigma; Poole, England) and was quantified as we previously described (O'Driscoll et al. (2008) Cancer Genomics Proteomics. 5(2):94-104). cDNA was synthesised using TaqMan microRNA reverse transcription kit (Applied Biosystems) and Multiplex RT Human Primer Pool Sets (8 primer pools/sample, each pool containing 48 different TaqMan reverse transcription primers). 100 ng total RNA was used for each of 8 RT reactions i.e. 800 ng/sample. Resulting cDNA was then diluted by a factor of 62.5, 50 μL of diluted cDNA was mixed with 50 μL of TaqMan universal PCR master mix (Applied Biosystems), and then 100 μL was added to the appropriate ports (8 ports/TaqMan low density array (TLDA) card, corresponding to 8 sets of cDNA/sample from 8 primer pools). TLDA cards were run on ABI 7900HT Real Time PCR system (Applied Biosystems). cDNA was applied to first generation arrays representing 48 human miRNAs. Following application of T-test, differentially expressed targets were identified as miRNAs with a fold change≧2 and p-value<0.05. The Results were as follows: Based on the criteria above, miR-146b-3p, miR-30c-11* and miR-616* were found to be 4.2 fold; 2.6 fold; and 6.9 fold higher levels in the Stage 1 ADC sera compared to the levels in the age- and gender matched controls.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

What is claimed is:

1. A diagnostic kit to detect lung adenocarcinoma, or to stratify patients according to expected prognosis comprising at least one oligonucleotide probe capable of binding to at least a portion of a circulating miRNA selected from the group comprising miR-556, -550, -939, -616*, -146b-3p and -30c-1*.

2. The kit of claim 1, wherein the group of circulating miRNAs further comprises miR-339-5p, and miR-656.

3. The kit as claimed in claim 2 adapted for performance of an assay selected from a real-time PCR assay, a micro-array assay, a histochemical assay or an immunological assay.

4. A method of identifying a therapeutic agent capable of preventing or treating lung adenocarcinoma, comprising testing the ability of the potential therapeutic agent to alter the expression of at least one circulating miRNA selected from the group comprising miR-556, -550, -939, -616*, -146b-3p and -30c-1*.

5. The method of claim 4, where the group of circulating miRNAs further comprises, miR-339-5p, and miR-656.

6. The method of claim 5 wherein the testing of the potential therapeutic agent comprises the use of a circulating miRNA selected from the group comprising miR-556, -550, -939, -616*, -146b-3p, -30c-1*, -339-5p, and -656 to detect lung adenocarcinoma, or to stratify patients according to expected prognosis or treatment regimen.

7. The method of claim 6 wherein the detection is carried out on a blood sample or a sample derived from blood.

8. A method of detecting or screening for lung adenocarcinoma, comprising analysing a sample of blood taken from a patient to determine a level in the sample of one or more circulating miRNAs selected from the group comprising miR-556, -550, -939, -616*, -146b-3p and -30c-1*, the level of at least one of the miRNAs in the sample indicating the presence of lung adenocarcinoma.

9. The method of claim 8, wherein a level of at least one of the miRNAs falling above a predetermined threshold value for that miRNA indicates the presence of lung adenocarcinoma.

10. The method of claim 9 wherein the predetermined threshold value is zero.

11. The method of claim 8, wherein the group of miRNAs further comprises miR-339-5p and miR-656.

12. The method of claim 11, wherein a level of a circulating miRNA selected from the group comprising miR-339-5p and miR-656 falling below a predetermined threshold value for that miRNA indicates the presence of lung adenocarcinoma.

13. The method of claim 5 or 11 comprising determining the level of at least 2 circulating miRNAs from the group.

14. The method of claim 5 or 11 comprising determining the level of at least 3 miRNAs from the group.

15. The method of claim 5 or 11 comprising determining the level of at least 4 miRNAs from the group.

16. The method of claim 5 or 11 comprising determining the level of at least 5 miRNAs from the group.
